(19) United States
(12) Patent Application Publication
(10) Pub. No.: US 2026/0083496 A1
(43) Pub. Date: Mar. 26, 2026

(54) APPLICATION OF STIMULATION TO ENABLE CIRCUMFERENTIAL ABLATION DURING RF ABLATION FOR RDN

(71) Applicant: Medtronic Ireland Manufacturing Unlimited Company, Dublin 2 (IE)

(72) Inventor: Gerry O. McCaffrey, Galway (IE)

(73) Assignee: Medtronic Ireland Manufacturing Unlimited Company, Dublin 2 (IE)

(21) Appl. No.: 19/112,193

(22) PCT Filed: Sep. 7, 2023
(86) PCT No.: PCT/EP2023/074555

§ 371 Date: Mar. 14, 2025

Related U.S. Application Data
(60) Provisional application No. 63/406,547, filed on Sep. 14, 2022.

(51) Int. Cl. A61B 18/14; A61B 18/00; A61B 18/02; A61B 18/12

(52) U.S. Cl. CPC A61B 18/1492; A61B 18/0218; A61B 18/1206; A61B 2018/0022; A61B 2018/00434; A61B 2018/00577; A61B 2018/00642; A61B 2018/00702; A61B 2018/0072; A61B 2018/0075; A61B 2018/00994; A61B 2018/0212; A61B 2018/0293; A61B 2018/1253; A61B 2018/126; A61B 2018/1467

(57) ABSTRACT

Systems and method for performing a therapeutic procedure including navigating a therapeutic device to target tissue, applying a first stimulus to the target tissue via a stimulus delivery element to contract the target tissue about the therapeutic device, and applying a first therapy to the target tissue contracted about the therapeutic device to denervate the target tissue.

This application claims the benefit of U.S. Provisional Patent Application Ser. No. 63/406,547, filed Sep. 14, 2022, the entire content of which is incorporated herein by reference.

### TECHNICAL FIELD

This disclosure is directed to systems and methods enabling positioning a therapeutic device within luminal tissues to form complete circumferential ablations during a therapeutic procedure. In particular aspects the disclosure is directed to methods and systems for denervating sympathetic nerves in or around vascular tissue.

### BACKGROUND

Catheters have been proposed for use with various medical procedures. For example, a catheter can be configured to deliver neuromodulation (e.g., denervation) therapy to a tissue site to modify the activity of nerves at or near the tissue site. The nerves can be, for example, sympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the sympathetic nerves in and around the renal, hepatic, splanchnic, and mesenteric arteries have been identified experimentally and in humans as a likely contributor to the complex pathophysiology of arrhythmias, hypertension, states of volume overload (e.g., heart failure), and progressive renal disease, and a variety of other disease states.

To address some of these disease states, percutaneous renal denervation has been developed and is one current methodology for treatment of disease states such as hypertension caused by over-activation of the sympathetic nerves found proximate the renal arteries. In one type of renal denervation procedure, a clinician delivers energy, such as radiofrequency to the wall of the renal arteries in an effort to sever the sympathetic nerves that are found in or near the tissue of the renal artery. Similar procedures have been developed using ultrasound, chemical, and cryogenic therapies. The goal of these therapies is to reduce or eliminate activity of sympathetic nerves surrounding the renal artery, which has been shown to be effective in the treatment of hypertension, without the side effects of other therapies. Despite the efficacy of renal and other artery denervation or neuromodulation procedures, improvements to the systems and methods employed in performing these procedures is always desired.

### SUMMARY

One aspect of the disclosure is directed to a method of performing a therapeutic procedure. The method includes navigating a therapeutic device to target tissue. The method also includes applying a first stimulus to the target tissue via a stimulus delivery element to contract the target tissue about the therapeutic device. The method also includes applying a first therapy to the target tissue contracted about the therapeutic device to denervate the target tissue. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method further including applying a second stimulus to the target tissue via the stimulus delivery element following the application of therapy to the target tissue. The method further including observing the application of the first and second stimulus to assess the contraction of the target tissue. The therapy may be applied following confirmation of contraction of the target tissue following application of the first stimulus. The method further including applying a second therapy when a contraction above a threshold is observed of the target tissue from the second stimulus. The first stimulus may be applied during the application of the first therapy. The method further including observing the application of the first stimulus and the first therapy, to assess the contraction of the target tissue. Relaxation of the target tissue below a threshold may indicate a successful denervation of the target tissue. A first stimulation modality is selected from the group including of bipolar or monopolar alternating current (AC), a direct current (DC), pulsed AC or DC pulses, biphasic pulses, multiphasic pulses, chemical, and combinations thereof. A first therapy modality is selected from the group including of monopolar or bipolar radio frequency (RD), microwave, ultrasound, chemical, cryogenic, and combination thereof. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a system for denervation of target tissue. The system includes an elongate catheter, a therapy delivery element operably associated with the elongate catheter, and a stimulus delivery element operably associated with the elongate catheter. The system includes a stimulation source in communication with the stimulus delivery element, where application of a stimulus via the at least one stimulus delivery element causes target tissue to contract on the elongate catheter to center the elongate catheter in the target tissue. The system also includes a therapy source in communication with the therapy delivery element, where application of the therapy denervates nerve tissue of the target tissue. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The system for denervation further including two stimulus delivery elements formed on the elongate catheter. The system for denervation further including a second stimulus delivery element formed on a guide catheter. The stimulus deliver element may also be formed on a guide wire. The therapy source and the stimulation source may also be a single electrical generator. A therapy source modality may be selected from the group including of monopolar or bipolar radio frequency (RF), microwave, ultrasound, chemical, cryogenic, and combination thereof. A stimulation source modality may be selected from the group including of bipolar or monopolar alternating current (AC), a direct current (DC), pulsed AC or DC pulses, biphasic pulses, multiphasic pulses, chemical, and combinations thereof. The system for denervation further including a shape memory portion configured to alter a shape of the elongate catheter to a helical configuration such that the therapy delivery element is in contact with the target tissue. The therapy element may be composed of one or more monopolar RF electrodes formed on the elongate catheter such that alteration of the shape contacts the electrodes on the target tissue in spaced relation to each other. The system for denervation may include a balloon wherein inflation of the balloon secures the elongate catheter relative to the target tissue. The therapy delivery element may be a plurality of RF electrodes formed on the balloon. The therapy delivery element may be is a microwave antenna within the balloon. The therapy deliver element may be a plurality of ultrasound transducers formed on the balloon. The therapy delivery element may be one or more needles for injection of a chemical or cryoablation medium to the target tissue. The system for denervation further including a computer including a memory storing thereon an application that when executed by a processor: causes the stimulation element to apply a stimulus proximate the target tissue to contract muscle fibers of the target tissue; causes the therapy element to apply therapy to the target tissue; and ceases application of the therapy to cease upon determination that the target tissue has been successfully denervated. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Further disclosed herein are systems and a method for performing a therapeutic procedure including navigating a therapeutic device to target tissue, applying a first stimulus to the target tissue via a stimulus delivery element to contract the target tissue about the therapeutic device, and applying a first therapy to the target tissue contracted about the therapeutic device to denervate the target tissue.

### BRIEF DESCRIPTION OF THE DRAWINGS

Various aspects and embodiments of the disclosure are described hereinbelow with references to the drawings, wherein:

FIG. 1 is a schematic diagram of a therapeutic system provided in accordance with the present disclosure;

FIG. 2 is a schematic view of a workstation of the therapeutic system of FIG. 1;

FIG. 3 is a perspective view of a therapeutic device of the therapeutic system of FIG. 1;

FIG. 4 is a perspective view of the therapeutic device of FIG. 3 shown advanced within a portion of the patient's anatomy and in a deployed condition;

FIG. 5 is a perspective view of the therapeutic device of FIG. 3 shown with tissue contracted about a portion of the therapeutic device;

FIG. 6 is a perspective view of another embodiment of a therapeutic device of the system of FIG. 1 provided in accordance with the disclosure;

FIG. 7 is a flow chart illustrating a method of performing a therapeutic procedure in accordance with the disclosure;

FIG. 8 is a flow chart illustrating a further method of performing a therapeutic procedure in accordance with the disclosure;

FIG. 9 is a flow chart illustrating yet a further method of performing a therapeutic procedure in accordance with the disclosure.

### DETAILED DESCRIPTION

This disclosure is directed to therapeutic systems and methods for denervation or neuromodulation of nerves such as the sympathetic nerves in and around blood vessels and other luminal tissues. To enhance the therapeutic effect the therapeutic system is configured to apply a stimulus to the blood vessels or other luminal tissues. The stimulus triggers nerves, including the sympathetic nerves in and around, for example, a blood vessel causing muscle tissues within the blood vessel to contract. The contraction of the muscle tissues in the blood vessel results in a change in diameter of the blood vessel. The change in diameter centers the therapeutic device (e.g., a catheter) in the blood vessel. The centering of the therapeutic device promotes good contact of the therapy applying portions (e.g., radiofrequency electrodes) on the inner walls of the blood vessel. As a result, with good contact of the therapy applying portions more complete therapies (e.g., complete circumferential ablations) can be achieved promoting more complete denervation of the sympathetic nerves beyond the blood vessel wall and resulting in more consistent and alleviation of a patient's disease state (e.g., reducing hypertension).

The therapeutic devices contemplated in this disclosure can apply one or more of a variety of therapeutic modalities. For example, the therapeutic modalities considered withing the scope of this disclosure include monopolar or bipolar radiofrequency, microwave, cryogenic, ultrasound, chemical, and other yet to be developed modalities. Any of these therapy modalities may be incorporated into a therapeutic device, such as a catheter, which is configured for navigation to a desired location within the patient. A catheter configured to deliver one or more of these therapeutic modalities may be percutaneously navigated, for example via the femoral artery, to reach the blood vessels of the aorta including the celiac artery, hepatic arteries, splanchnic arteries, mesenteric arteries, renal arteries, and others that are enervated with sympathetic nerves or are proximate one or more sympathetic nerve ganglia. Such a catheter may also be laparoscopically placed in one or more of the above-identified blood vessels, or another luminal tissue without departing from the scope of this disclosure. Further, when utilized in connection with balloon catheter embodiments, the contraction of the muscle tissues in the blood vessels enables the use of a standard size balloon to be employed in different size blood vessels. As described in greater detail below, the balloon is a non-compliant balloon, that is its diameter, once inflated, does not change as a result of the application of pressure from outside the balloon (e.g., from contraction of a blood vessel in which the balloon is placed). As a result, the contraction ensures that the balloon, and particularly the therapy elements (e.g., electrodes) formed thereon make good contact with the blood vessel wall and enable consistent circumferential ablations in accordance with this disclosure.

The therapeutic devices are also configured to deliver stimulation to the blood vessel or other luminal tissue. This stimulation can take a variety of forms including electrical muscle stimulation (EMS) and chemical stimulation (e.g., local application of a vasoconstricting drug). The EMS energy may be one or more of direct current (DC), alternating current (AC), pulsed AC or DC and combinations of both. In certain aspects, described in greater detail below, the stimulation energy is biphasic or multiphasic pulsed DC. The amplitude, frequency, pulse width, and duration of stimulation energy can be selected to ensure contraction of the muscle tissues of the luminal tissue without damaging the luminal tissue, the muscles, or the nerves triggering the contraction. Where chemical stimulation employed, the vasoconstrictor may be selected for example from epinephrine, norepinephrine, phenylephrine, and others known to those of skill in the art, and the dose may be selected to achieve an acute constriction of a specific time frame or a continued construction that is alleviated upon completion of the denervation therapy.

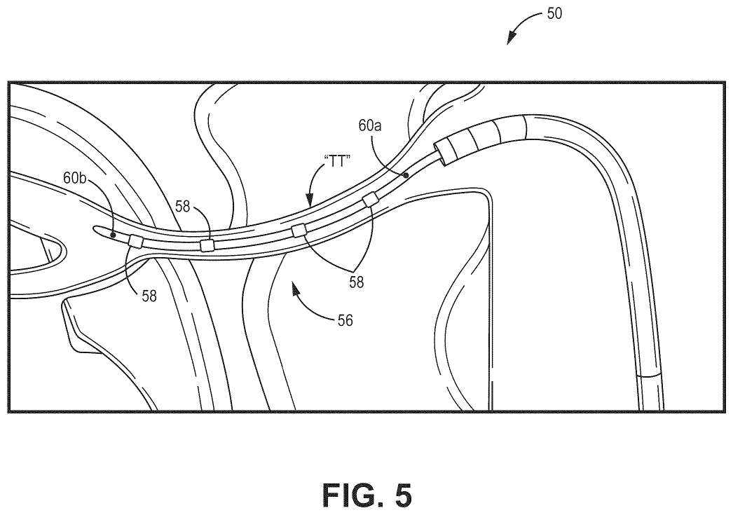

As described above, one of the goals of the stimulation is to contract the blood vessel or other luminal tissue around a therapy applying portions of the therapeutic device 50 as depicted in FIG. 5. In instances where the therapeutic device includes multiple therapy applying portions (e.g., multiple RF electrodes), the therapy applying portions may be disposed in spaced relation to one another. In addition, where separate stimulation applying portions are employed, the stimulation applying portions may also be in spaced relation from both each other and from the therapy applying portions. For example, where the therapy applying portions are between two stimulation applying portions, the contraction of the blood vessel or other luminal tissue promotes contact of the therapy applying portions on the blood vessel or other luminal tissue wall.

With the tissue constricted about the therapy applying portions of the therapeutic device, the therapeutic device is centered within the blood vessel or other luminal tissue such that the delivery of the therapy achieves a desired ablation pattern (e.g., circumferential ablation) to ensure complete denervation of the sympathetic nerves around the blood vessel or other luminal tissue. of the target tissue.

A variety of options are contemplated for relative placement of the therapy applying portions and the stimulation applying portions of the therapeutic device. As noted above, two or more stimulation portions may be placed such that the therapy applying portions are between them. Alternatively, multiple stimulation portions may be employed and interspersed between the therapy applying portions. Still further, a single stimulation applying portion may be employed. In a further embodiment, one or more of the stimulation-applying portions may be disposed on a separate component from the therapeutic device, such as a guide wire, a guide sheath, a second catheter, etc.

The therapeutic device is coupled to a therapy source and a stimulation source although it is envisioned that the therapy source and the stimulation source may be the same and capable of generating both therapy and stimulation. For example, an electrical generator may be configured to generate biphasic or multiphasic DC pulses to be supplied to the stimulation applying portions of the therapeutic device and to supply monopolar RF energy to the therapy applying portions. Additionally or alternatively, the modalities for therapy and stimulation may be similar, as described above, or very different such as chemical stimulation (local vasoconstrictor application) and cryoablation therapy. Any combination of the above-described therapy modalities and stimulation modalities is contemplated within the scope of the disclosure.

In accordance with aspects of the disclosure the therapeutic device may be navigated in one configuration (e.g., linear) and once at a desired location deployed or actuated to achieve a second configuration (e.g., expanded balloon or shape memory helical shape). The second configuration may be achieved either before or after application of the stimulus. Regardless of when applied, in accordance with this disclosure the contraction of the blood vessel or other luminal tissue acts on the therapeutic device and may place pressure on the therapeutic device potentially altering the shape of the therapeutic device. As noted above, this contraction of the blood vessel or luminal tissue ensures close contact of the therapy applying portions and an inner wall of the blood vessel or luminal tissue so that the efficacy of the therapy can be ensured. Application of the stimulation may also be performed multiple times before, during, and after application of therapy. Still further, the application of the stimulation may be employed to assess the effectiveness of the denervation procedure. Following a successful denervation application of a stimulation may result in limited or no contraction of the blood vessel or other luminal tissue as the nerves which stimulate the muscle tissue of the blood vessel or luminal tissue have been severed and are unable to trigger contraction of the muscle tissue.

For ease of description, much of the following description focuses on implementations of electrical stimulation and RF denervation. Though those of skill in the art will recognize that the methods and systems described herein may employ any of the therapy and stimulation modalities described herein. Similarly, the following description focuses on navigation to and application of stimulation and therapy to the renal artery to denervate sympathetic nerves in, around, and proximate the renal arteries. However, the disclosure is not so limited and can be employed for denervation nerves accessible via any blood vessel or other luminal tissue (e.g., a bile duct).

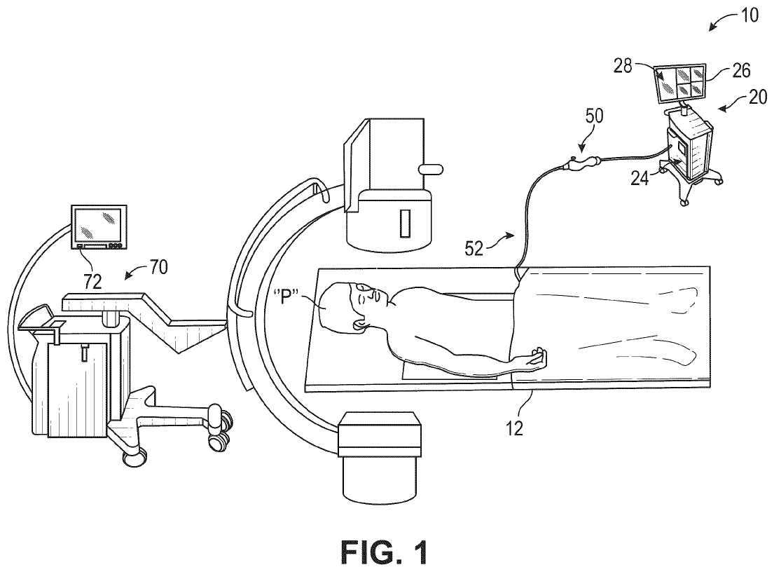

Turning now to the drawings, FIG. 1 illustrates a therapy system 10 in accordance with the present disclosure. As will be described in further detail hereinbelow, the therapy system 10 enables navigation of the therapeutic device 50 to a desired location within the patient's anatomy (e.g., the patient's renal artery), deliver stimulation to the renal artery causing its contraction and denervating the sympathetic nerves in and around the renal artery.

The therapy system 10 includes a workstation 20, a therapeutic device 50 operably coupled to the workstation 20, and an imaging device 70, which in may be operably coupled to the workstation 20. The patient “P” is shown lying on an operating table 12 with the therapeutic device 50 inserted through a portion of the patient's femoral artery, although it is contemplated that the therapeutic device 50 may be inserted into any suitable portion of the patient's vascular network that is in fluid communication with a desired blood vessel for therapy.

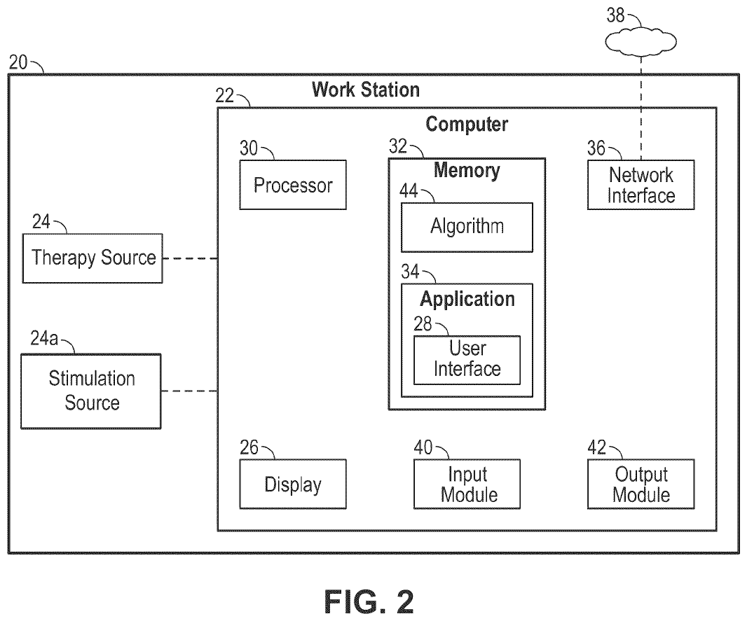

Continuing with FIG. 1 and with additional reference to FIG. 2, the workstation 20 includes a computer 22, a therapy source 24 (e.g., RF generator, microwave generator, ultrasound generator, cryogenic medium source, chemical source, etc.) operably coupled to the computer 22, and a stimulation source 24a operably coupled to the computer 22. Although generally described as being separate from the therapy source 24, it is envisioned that the stimulation source 24a may be integrated within the therapy source 24, as described above, and the therapy source 24 may generate both therapy and stimulation modalities.

The computer 22 is coupled to a display 26 that is configured to display one or more user interfaces 28. The computer 22 may be a desktop computer or a tower configuration with display 26 or may include a laptop computer or other computing device. The computer 22 includes a processor 30 which executes software stored in a memory 32. The memory 32 may store one or more applications 34 and/or algorithms 44 to be executed by the processor 30. A network interface 36 enables the workstation 20 to communicate with a variety of other devices and systems via the internet. The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, and/or videos may be stored. The cloud storage system 38 may be remote from or on the premises of the hospital such as in a control or hospital information technology room. It is envisioned that the cloud storage system 38 could also serve as a host for more robust analysis of acquired images (e.g., fluoroscopic, CT, MRI, CBCT, etc.), data, etc. (e.g., additional or reinforcement data for analysis and/or comparison). An input module 40 receives inputs from an input device such as a keyboard, a mouse, voice commands, a therapy source controller (e.g., a foot pedal or handheld remote-control device that enables the clinician to initiate, terminate, and optionally, adjust various operational characteristics of the therapy source 24 and/or stimulation source 24a, including, but not limited to, power delivery), amongst others. An output module 42 connects the processor 30 and the memory 32 to a variety of output devices such as the display 26. In embodiments, the display 26 may be a touchscreen display.

The therapy source 24 generates and outputs one or more of RF energy (monopolar or bipolar), microwave energy, ultrasound energy, cryogenic medium, or chemical ablation medium via an automated control algorithm 44 stored on the memory 32 and/or under the control of a clinician. As can be appreciated, the therapy generated or output by the therapy source 24 changes a temperature of the tissue (e.g., increases or decreases the temperature) to achieve the desired denervation of the sympathetic nerves. The therapy source 24 may be configured to produce a selected modality and magnitude of energy and/or therapy for delivery to the treatment site via the therapeutic device 50, as will be described in further detail hereinbelow. The therapy source 24 may monitor voltage and current applied to target tissue via the therapeutic device 50 and monitors the temperature of the target tissue or tissue proximate the target tissue, and/or a portion of the therapeutic device 50.

The stimulation source 24a generates and delivers stimulation, including one or more of DC, AC, pulsed AC or DC (e.g., biphasic or multiphasic pulsed DC) and combinations of these via the automated control algorithm 44 stored on the memory 32 or as directed by a clinician. Similarly, the stimulation source may store cryogenic or chemical medium to apply those modalities of stimulation. As will be appreciated, the stimulation generated and/or delivered by the stimulation source 24a over a period of time effectuates contractions of the muscle tissue of the blood vessel or other luminal tissue to reduce the diameter. The stimulation generated and/or delivered by the stimulation source 24a is of a magnitude or volume to ensure that tissue is not damaged, but merely that the nerves are stimulated to achieve the desired muscular contraction and reduction in blood vessel or luminal tissue diameter. As noted above, the stimulation source 24a may deliver stimulation that is of a different modality than the therapy delivered by the therapy source 24.

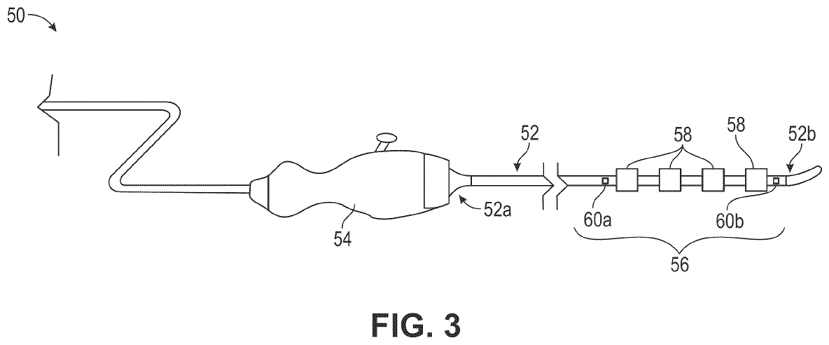

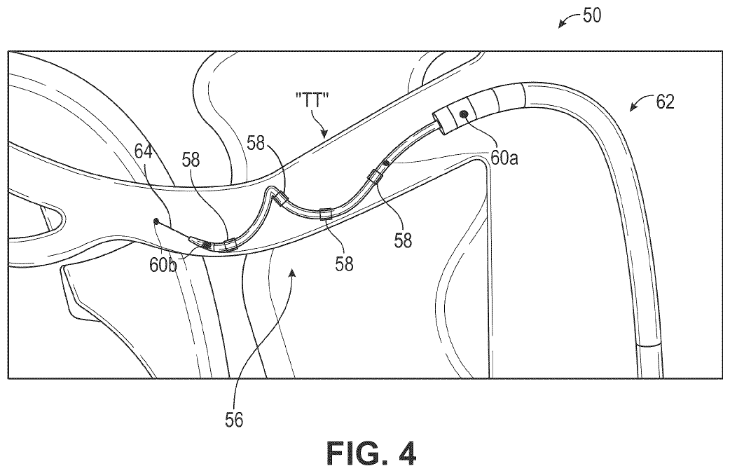

FIGS. 3 and 4 depict one embodiment of a therapeutic device 50 in accordance with the disclosure. The therapeutic device 50 includes an elongated shaft 52 having a handle 54 disposed on a proximal end portion 52a of the elongated shaft 52. The therapeutic device 50 includes a therapeutic portion 56 at which therapy delivery elements such as RF electrodes 58 as shown in FIG. 3, are located. The elongated shaft 52 of the therapeutic device 50 is configured to be advanced within a portion of the patient's vasculature, such as a femoral artery or other suitable portion of patient's vascular network that is in fluid communication with the patient's renal artery. In embodiments, the therapeutic portion 56 is configured to be transformed from an initial, undeployed state having a generally linear profile (FIG. 3), to a second, deployed or expanded configuration, where the therapeutic portion 56 forms a generally spiral and/or helical configuration (FIG. 4) for delivering therapy at the treatment site and providing therapeutically-effective electrically and/or thermally induced renal neuromodulation. In this manner, when in the second, expanded configuration, the therapeutic portion 56, and particularly the individual electrodes 58, is pressed against or otherwise contacts the walls of the patient's vasculature tissue. Although generally described as transitioning to a spiral and/or helical configuration, it is envisioned that the therapeutic portion 56 may be deployed in other configurations without departing from the scope of the disclosure. Further, the therapeutic device 50 may be configurable, for example using one or more pull wires (not shown) to adjust the configuration to promote contact between the electrodes 58 and the wall of the renal artery. As such the therapeutic device 50 may be capable of being placed one, two, three, four or more different configurations depending upon the design needs of the therapeutic device 50 or the location at which therapy is to be applied.

FIGS. 3 and 4 are described here in connection with an RF therapeutic device employing electrodes 58. Those of skill in the art will recognize that the electrodes 58 may be replaced with ultrasound transducers, ports and/or extending needles for cryogenic and chemical ablation medium, or microwave ablation antennae as described elsewhere herein and without departing from the scope of the disclosure.

As depicted in FIG. 4, the elongated shaft 52 is configured to be received within a guide catheter 62 (e.g., a 6F guide catheter) that is utilized to navigate therapeutic device 50 to a desired location at which point if a guide catheter 62 is retracted to uncover the therapeutic portion 56 of the therapeutic device 50. As noted above, retraction of the guide catheter 62 may enable the therapeutic portion 56 to transition from the first, undeployed state, to the second, deployed or expanded state.

The elongated shaft 52 of the therapeutic device 50 may further include an aperture (not shown) that is configured to slidably receive a guidewire over which the therapeutic device 50, either alone or in combination with the guide catheter 62 are advanced. In this manner, the guidewire is utilized to guide the therapeutic device, to the target tissue using over-the-wire (OTW) or rapid exchange (RX) techniques, at which point the guide wire 64 may be partially or fully removed from therapeutic device 50, enabling transitioning of the therapeutic device 50 from a first, undeployed state (FIG. 3), to the second, deployed or expanded state (FIG. 4). As noted elsewhere herein the therapeutic portion 56 may be transitioned from the first, undeployed state to the second, deployed state automatically (e.g., via a shape memory alloy, etc.) or manually (e.g., via pull wires controlled by the clinician).

Continuing with FIGS. 3 and 4, in embodiments where the therapeutic device 50 is a RF ablation catheter, the therapeutic portion 56 includes one or more electrodes 58 disposed on an outer surface thereof that are configured to contact a portion of the patient's vascular tissue when the therapeutic device 50 is placed in the second, expanded configuration. Though described herein below with respect to RF modalities, one of skill in the art will recognize that the electrodes 58 may be replaced with ultrasound transducers, microwave antennae, ports for delivery of cryoablation medium or chemical medium and other implements known to additional ablation and denervation modalities without departing from the scope of the disclosure. As shown herein, the therapeutic device 50 includes four electrodes 58. However, the disclosure is not so limited and the therapeutic device 50 may have more or fewer electrodes 58 (or other modality therapy delivery element) without departing from the scope of the disclosure.

As shown in the figures, the electrodes 58 are disposed in spaced relation to one another along a length of the therapeutic device 50 forming the therapeutic portion 56. As will be appreciated these electrodes 58 (or any other modality therapy delivery element) will be in communication with the therapy source 24. As shown in the electrodes 58 are in electrical communication with the therapy source 24 which produces monopolar RF energy to denervate the sympathetic nerves of the relevant blood vessel. It is envisioned that the therapy delivery elements 58 may deliver therapy independently of one another (e.g., monopolar), simultaneously, selectively, sequentially, and/or between any desired combination of the electrodes 58 (e.g., bipolar). Further, in at least one aspect of the disclosure the electrodes 58 are employed to deliver the stimulation to the blood vessel in question. Where electrical energy is employed for the stimulation, the energy (e.g., biphasic or multiphasic DC pulses) is generated by the stimulation source 24a and communicated to the electrodes 58 causing the muscle reactions described herein above and the contraction of the blood vessel.

Additionally, or alternatively, the therapeutic device 50 may include one or more stimulation delivery elements 60 disposed on the therapeutic device 50. In accordance with one aspect of the disclosure employing bipolar stimulation, stimulation energy from the stimulation source 24a is generated and applied to tissue of a relevant blood vessel or luminal tissue. The stimulation is applied to a first stimulation delivery element 60a and transmitted to stimulation delivery element 60b. In the process, the signal produces by the stimulation source (e.g., a biphasic or multiphasic pulsed DC signal) is transmitted through the tissue of the blood vessel and triggers one or more nerves (e.g., sympathetic nerves) in or proximate the blood vessel causing muscle tissue in the blood vessel to contract ensuring that the electrodes 58 are in contact with the wall of the blood vessel and in a desired position for application of a therapy (e.g., a denervation). As shown the first stimulation delivery element 60a is disposed proximal of a proximal most of the electrodes 58 and the second stimulation delivery element 60b is disposed distal of a distal most electrode such that the four shown electrodes 58 are between the first and second stimulation delivery elements 60a, 60b (e.g., bookended by the first and second stimulation delivery elements 60a, 60b).

In view of this configuration, the stimulation energy (e.g., biphasic or multiphasic DC pulses), transmits through the blood vessel between the first and second stimulation delivery elements 60a, 60b causing the blood vessel to contract at least along that length bringing the inner wall of the blood vessel into the desired contact with the electrodes 58 prior to commencement of the therapy. Continued application (e.g., a train of biphasic or multiphasic pulses) may be employed to ensure continued contraction of the blood vessel ensuring continued desired contacts with the electrodes 58 during the therapy application.

In some instances with the therapeutic device 50 in the second or helical configuration, force applied to the therapeutic portion 56 by the constricting muscle tissue of the blood vessel causes a length of the therapeutic device 50 to linearly increase (e.g., along a length of the tissue) to achieve a third configuration (e.g. a desired configuration for application of therapy), causing a greater and more uniform contact between the tissue of the inner wall of the blood vessel and the electrodes 58. With the therapeutic device 50 in the third configuration, the therapy delivered by may be better received by the tissue, and particularly the sympathetic nerves to achieve a circumferential therapy pattern around the blood vessel ensuring that regardless of where along the blood vessel the sympathetic nerve is found, it has been severed. In addition, the circumferential pattern has a longitudinal offset which helps ensure that the tissues of the blood vessel, including the muscle, do not receive so much therapy that necrosis results in any portion of the blood vessel.

As can be appreciated, if monopolar stimulation is being applied to the blood vessel via the stimulation delivery elements 60a or 60b, either may be replaced with an electrode return pad (not shown). The return pad may be utilized in addition to the first and second stimulation delivery elements 60a, 60b or as an alternative to one of the first or second stimulation delivery elements 60a, 60b.

It may be necessary to apply stimulation to the tissue multiple times depending upon the amount of constriction of the tissue about the therapeutic device 50 or the efficacy of the therapy delivered. In this manner, if it is determined that the tissue has not contacted about the therapeutic device 50 as desired, stimulation may be applied one or more additional times until the contraction or amount of contraction is achieved. Contraction of the blood vessel or luminal tissue onto the therapeutic device 50 may be confirmed with intra-procedure imaging such as fluoroscopy, though other imaging modalities may be employed including ultrasound, computed tomography, cone-beam computed tomography, MRI and others. As shown in FIG. 1 the imaging device 70 (e.g., fluoroscopic)is operably coupled to a display 72 and/or to the workstation 20. Navigation and placement of the therapeutic device 50 at a desired location can be observed using the output of the imaging device. To assist in observation, the therapeutic device 50 or for example the electrodes 58 may be formed from or coated with a radiopaque material to improve their resolution in the images.

Radio opaque dyes injected into the patient P assist in observing the contraction of the blood vessel or luminal tissue. By imaging the blood vessel prior to the application of stimulation and after application (e.g., observation under live fluoroscopy) the clinician can observe the effects and decide whether more stimulation is required or whether therapy can commence. To further assist in the effects of stimulation, the therapeutic device 50 may include one or more sensors, such as pressure sensors, fiber Bragg grating, strain gauges, etc. (not shown) to provide further indications of the contraction of the muscle tissues of the blood vessel or luminal tissue. The contraction measured by the one or more sensors may be compared to one or more predetermined thresholds and an indicator may be provided on the user interface 28 in the display 26 of the computing device 20 indicating to the clinician that sufficient contraction has been observed and that therapy can commence, or that insufficient contraction has been observed and further stimulation or placement adjustment may be advised before therapy application.

Duration of the stimulation and therapy may either be pre-programmed and stored in a control algorithm 44 stored on the computing device 20 or may be manually applied and adjusted based on the live imaging described above. Where the stimulation and therapy are application driven, these may also be dynamic based on feedback from the one or more sensors on the therapeutic device 50. For example, the magnitude and duration of the stimulation may be altered until there is an observation of adequate contraction of the muscle tissue. In one embodiment, the stimulation is applied repeatedly during the procedure to ensure contraction of the muscle tissue, therapy is applied while stimulation is also being applied (or at least with the muscle tissue contracted) and when it is observed that despite the continued stimulation the blood vessel or luminal tissue no longer contracts, the procedure may be terminated. This failure to contract signifies that the nerves, particularly the sympathetic nerves have been denervated and that the procedure was successful. As will be appreciated, this process may be bounded by one or more safety measures, such observation of total energy delivered, blood vessel wall temperature and others to ensure that even if a successful denervation is not achieved the patient and the tissues being treated are not unnecessarily harmed. A look-up table or data of predetermined thresholds may be saved within the memory 32 and accessed by the control algorithm 44 or other suitable application stored on the memory during the procedure and alerts may be presented on the user interface 28.

Though not shown, the therapeutic device 50 may be operably coupled to an irrigation source (not shown). The irrigation source can cool the blood vessel wall during therapy application to prevent necrosis. In addition, the irrigation can provide a medium through which the stimulation can traverse in instances where the contraction of the blood vessel has reduced the blood flow. The irrigation flow from the guide catheter 62, or through one or more lumen formed within the therapeutic device 50 to arrive at the treatment site.

Previously described herein, the stimulation portions 60a, 60b are formed on the therapeutic device 50. Alternatively, in one or more embodiments, the electrodes 58 may be employed to deliver the stimulation and themselves connected to the stimulation source 24a. however, the disclosure is not so limited and in addition to the above, one or more of the stimulation portions may be formed on the guide catheter 62 or on a guide wire 64 over which the therapeutic device 50 is navigated to a desired location. Further, stimulation may in some embodiments be delivered by a separate tool either navigated to the desired location internally or capable of application of stimulation from outside the body of the patient to achieve the desired contraction of the blood vessel or luminal tissue.

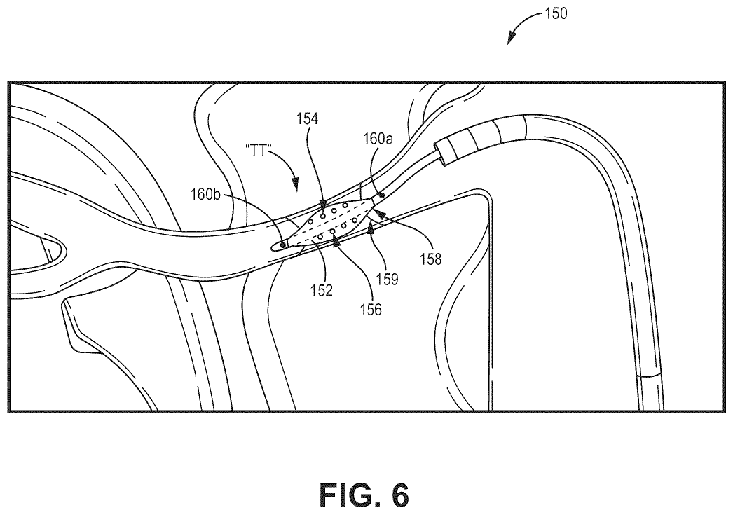

Heretofore the therapeutic device 50 has been primarily described in connection with a shape memory construction where exit from a guide catheter 62 or withdrawal of a guide wire frees the shape memory alloy to achieve a desired spiral shape. As noted elsewhere, however, the disclosure is not so limited. With reference to FIG. 6, a therapeutic device 150 may employ a balloon 152. As above, during a navigation phase, the balloon 152 has a generally linear shape, and when at a desired location, the balloon 152 can be expanded to achieve a desired shape. The desired shape may be based on the size of the blood vessel or luminal tissue into which the therapeutic device 150 is to be navigated and to which therapy is to be applied. As is known in the art balloons may be employed to ensure contact of RF electrodes 154 and ultrasound transducers 156 against tissue. Further, they may be employed to center a microwave antenna 158 in the balloon 152.

In some aspects of the disclosure, the balloon 152 may be formed of an inelastic material such that once inflated or deployed (e.g., with saline or another liquid) to its designed pressure the balloon 152 will not stretch, elongate, or otherwise change its shape when a force is applied to the exterior of the balloon 152. In some embodiments the balloon 152 is configured with a standard inflated or deployed diameter. The use of a single diameter enables a standardized product to be utilized regardless of where the ablation is to be undertaken (e.g., renal artery, hepatic artery, mesenteric artery, etc.). Inflation or deployment of the balloon 152, followed by application of stimulation energy causes contraction of the muscles of the blood vessel ensuring contact of the blood vessel onto the balloon 152 and centering of the balloon 152 in the blood vessel. Once in this state, the application of therapy results in a circumferential ablation being formed around the balloon 152 and on the blood vessel, as described elsewhere herein.

In connection with the above, the balloon may be multi-chambered or have one or more fluid ports allowing for example for different medium to flow into the balloon or different chambers of the balloon. In one example, the balloon 152 may be inflated with saline that surrounds the microwave antenna 158 allowing for good coupling of the microwave energy emitted from the antenna to the tissue receiving therapy. The saline also works to sub cool the tissue closest to the antenna (e.g., the blood vessel wall) to prevent necrosis of this tissue. The balloon may also be used to limit the extent a cryogenic medium can act on tissues limiting the spread of the cryogenic medium to just the area of the balloon 152. In cryogenic and chemical ablations, the therapeutic device 150 may include one or more needles 159 that can project from the therapeutic device 150 and are fluidly connected to a therapy source 24 supplying these therapies directly to the desired tissue. The balloon 152 in such cases provides a centered and stable platform for the needles 159 to exit the therapeutic device 150 and enter the tissue. In connection with the disclosure the balloon 152 may be an occlusive balloon, a non-occlusive balloon, or another configuration of a balloon permitting the flow of blood, or other media through the blood vessel or luminal tissue.

In connection with the above, the therapeutic device 50, 150 may include multiple modalities for therapy such that for example a single device therapeutic device may include RF electrodes 154 and ultrasound transducers 156, or RF electrodes 154 and chemical ablation needles 159. Further, each of these is connected to a therapy source 24 configured to supply the denoted therapy type. Those of skill in the art will recognize that the therapeutic device 150, and the therapy source 24 may provide any suitable combination of therapies capable of performing a denervation procedure.

Like the embodiments of FIGS. 3 and 4, the therapeutic device 150 may include one or more stimulation delivery elements 160 disposed thereon. In an embodiment where bipolar stimulation is applied to the tissue, the therapeutic device 150 may include a pair of stimulation delivery elements 160a and 160b. A first stimulation delivery element 160a may be disposed proximal of the balloon 152 and the second stimulation delivery element 160b may be disposed distal of balloon 152 such that the balloon 152 and any therapy delivery element disposed therein or thereon is interposed between the first and second stimulation delivery elements 160a, 160b. As will be appreciated, in instances where RF electrodes 154 are placed on the balloon 152, the electrodes 154 may themselves act as the stimulation delivery elements and require no further elements to provide the stimulation. Similarly, the stimulation delivery elements may be located on a guide wire 64 a guide catheter 62 or a separate device, as described elsewhere herein.

As with other embodiments, application of stimulation causes the blood vessel or luminal tissue to contract centering the therapeutic device 150. The contraction may cause the balloon 152 to change shape, and in some embodiments a slight deflation may be enabled. Regardless, the contraction helps to center the therapeutic device 150 in the blood vessel or luminal tissue and promotes placement of RF electrodes 154, ultrasound transducers 156, against tissue at desired locations, promotes centering of a microwave antenna 158 and cryogenic features in the blood vessel or positioning of needles 159 so that they can be effectively deployed for chemical therapy application.

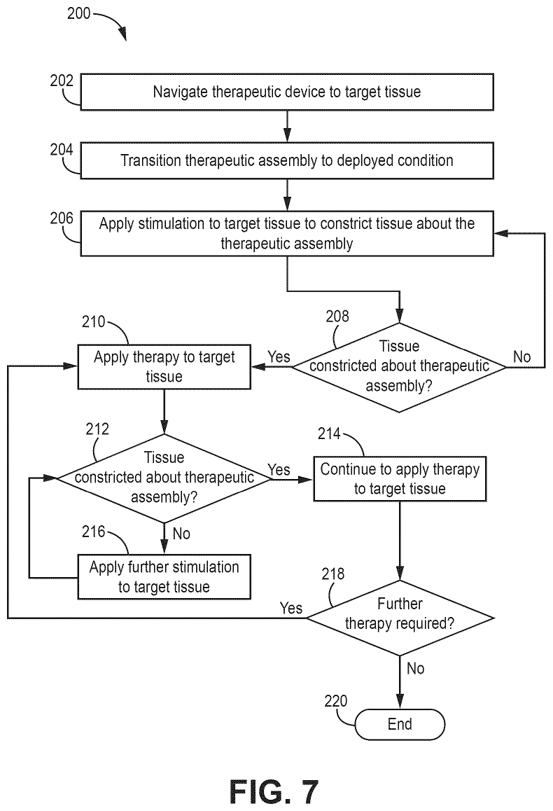

With reference to FIG. 7, a method of performing a therapeutic procedure is illustrated and generally identified by reference numeral 200. In step 202, the therapeutic device 50 is navigated to the target tissue. Once the therapeutic device 50 is located adjacent the target tissue, the therapeutic portion may optionally be transitioned from a first state to a second deployed state, in step 204, such that the one or more therapy delivery elements abut or otherwise contact the tissue. In step 206, stimulation is applied to the target tissue via stimulation delivery elements (e.g., 60a, 60b) to cause the blood vessel or luminal tissue to contract about the therapeutic device 50, 150. In step 208, it is determined if the blood vessel or luminal tissue has contracted about the therapeutic device 50 and/or whether such constriction is sufficient. If the blood vessel or luminal tissue has not contracted about the therapeutic device 50 or further contraction is required, the method returns to step 206 and further stimulation is applied. If the tissue has contracted about the therapeutic device 50, in step 210, therapy is applied to the blood vessel or luminal tissue. In step 212, during the application of therapy to the tissue, it is determined if the blood vessel or luminal tissue remains is constricted about the therapeutic device 50 or if further stimulation is required to maintain contraction of the tissue about the therapeutic device 50. If it is determined if the amount of contraction is adequate, application of therapy is continued in step 214. If the tissue has relaxed and/or contraction of the tissue is no longer adequate, in step 216, further stimulation is applied to the tissue and the method returns to step 212. In step 218, it is determined whether the application of therapy to the tissue resulted in the desired outcome. If further therapy is required, the method returns to step 210. If no further therapy is required, the method ends at step 220. Those having skill in the art will recognize that the steps of method 200 may be repeated as many times as necessary, depending upon the needs of the therapeutic procedure being performed.

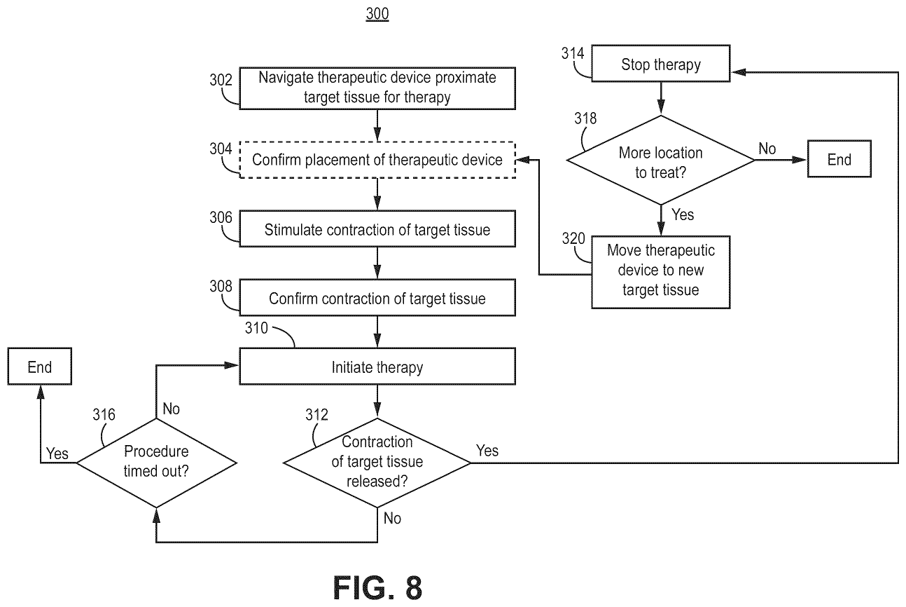

A further embodiment of the disclosure is depicted in connection with FIG. 8. The method 200 is described as one where stimulation may be intermittently applied during the procedure. In contrast, method 300 is one where stimulation is continuously applied to the target tissue (e.g., a blood vessel or other luminal tissue) during the procedure and the cessation of contraction of the target tissue signals a successful therapy (e.g., denervation of the sympathetic nerves achieving the contraction). Method 300 starts at step 200 where the therapeutic device 50/150 is navigated to a location proximate the target tissue (e.g., renal artery, hepatic artery, etc.) At step 304, the placement of the therapeutic device proximate the target tissue may be optionally confirmed. For example, step 304 may be done using fluoroscopy or other imaging modalities. Those of skill in the art will recognize that the imaging of the target location may be performed throughout the procedure and employed for the placement confirmation. Once the therapeutic device 50/150 is located proximate the target tissue, at step 306 stimulation is applied to the target tissue to achieve contraction of the muscle fibers, as described above. Using either fluoroscopy or another imaging modality or one or more sensors as described above, at step 308 contraction of the target tissue is confirmed. Once contraction of the target tissue is confirmed therapy is initiated at step 310. The therapy may include application of one or more of monopolar or bipolar RF, microwave, ultrasound, cryogenic, chemical, or other modalities as described elsewhere herein. Once therapy is initiated, the contraction of the target tissue is observed, for example using fluoroscopic imaging and when at step 312 the contraction that was confirmed in step 308, the method proceeds to step 314 where the therapy is stopped. If no release of the contraction is observed, either via fluoroscopic imaging or via one or more sensors, is observed at step 312, the method proceeds to step 316 where a safety check is undertaken. The safety check 316 may be as simple as a check to ensure that a therapy time limit is not exceeded, that an amount of energy delivered is note exceeded, or some other parameter is not exceeded. If the procedure has not timed out (or exceeded some other safety parameter) the method 300 returns to step 310 where therapy continues. If a safety parameter, such as the procedure duration has been exceeded at step 316, the procedure ends.

Following stopping of the therapy at 314, a determination is made whether additional treatment locations remain to receive therapy. If no, the procedure ends, however, if one or more treatment sites remain to receive therapy the therapeutic device is moved to one of those locations at step 320 and the process returns to the optional step 304 where placement is confirmed. The procedure continues till all tissue sites have received the desired therapy.

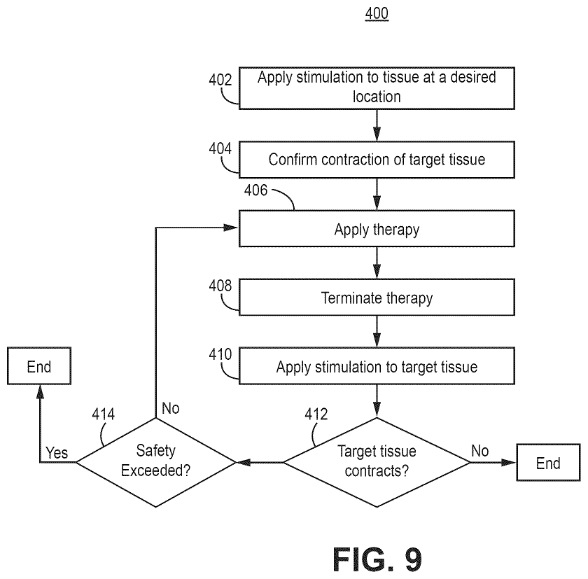

FIG. 9 depicts a further embodiment of the disclosure. FIG. 9 depicts a method 400 for application of a therapy to a desired location in accordance with the disclosure. At step 402 stimulation is applied to target tissue. Following application of the stimulation contraction of the target tissue is confirmed at step 404, for example via fluoroscopic imaging. At step 406 therapy is applied. The therapy may be for example algorithm-based wherein an algorithm 44 on computer 22 controls the application of therapy to the target tissue. Alternatively, the application of therapy may be manually controlled by the clinician. At step 408 the therapy is terminated, either manually or via the algorithm 44. Following application of the therapy, stimulation is applied at step 410 to the target tissue. At step 412 an assessment is made whether the target tissue contracts as a result of the stimulation applied at step 410. Step 412 may employ imaging such as fluoroscopic imaging or the use of one or more sensors as described herein. If the target tissue no longer contracts, then the procedure can end. The failure of the target tissue to contract can be understood to indicate that the denervation of nerves, such as the sympathetic nerves of a blood vessel, has been successful. If, however, at step 412 contraction of the target tissue is observed (e.g., via sensors or imaging), the method moves to step 414 where a check is made to ensure that no safety parameter has been exceeded. The safety parameter may be for example, an assessment of total duration of the procedure, the total energy delivered or removed from the tissue, or other parameters which if exceeded could undesirably damage tissues proximate the target tissue. If any of these have been exceeded, method 400. If no safety parameter has been exceeded at step 414, the method returns to step 406 for further application of therapy. In this manner therapy can be applied until a successful denervation has been achieved.

Although described generally hereinabove, it is envisioned that the memory 32 may include any non-transitory computer-readable storage media for storing data and/or software including instructions that are executable by the processor 30 and which control the operation of the workstation 20 and, in some embodiments, may also control the operation of the therapeutic device 50, imaging device 70, etc. In an embodiment, the memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the therapy source 24.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

The following examples are a non-limiting list of clauses in accordance with one or more techniques of this disclosure.

Example 1. A method of performing a therapeutic procedure, comprising: navigating a therapeutic device to target tissue; applying a first stimulus to the target tissue via a stimulus delivery element to contract the target tissue about the therapeutic device; and applying a first therapy to the target tissue contracted about the therapeutic device to denervate the target tissue.

Example 2. The method according to Example 1, further comprising applying a second stimulus to the target tissue via the stimulus delivery element following the application of therapy to the target tissue.

Example 3. The method according to Example 2, further comprising observing the application of the first and second stimulus to assess the contraction of the target tissue.

Example 4. The method according to Example 3, wherein the therapy is applied following confirmation of contraction of the target tissue following application of the first stimulus.

Example 5. The method according to Example 3, further comprising determining a successful denervation when no contraction or contraction below a threshold of the target tissue is observed from the second stimulus.

Example 6. The method according to Example 3, further comprising applying a second therapy when a contraction above a threshold is observed of the target tissue from the second stimulus.

Example 7. The method according to Example 1, wherein the first stimulus is applied during the application of the first therapy.

Example 8. The method according to Example 7, further comprising observing the application of the first stimulus and the first therapy, to assess the contraction of the target tissue.

Example 9. The method according to Example 8, further comprising stopping application of the first therapy upon observing a relaxation of the target tissue contracted by the application of the first stimulus, wherein relaxation of the target tissue indicates a successful denervation of the target tissue.

Example 10. The method according to Example 1, wherein a first stimulation modality is selected from the group consisting of bipolar or monopolar alternating current (AC), a direct current (DC), pulsed AC or DC pulses, biphasic pulses, multiphasic pulses, chemical, and combinations thereof.

Example 11. The method according to Example 1, wherein a first therapy modality is selected from the group consisting of monopolar or bipolar radio frequency (RF), microwave, ultrasound, chemical, cryogenic, and combination thereof.

Example 12. A system for denervation of target tissue comprising: an elongate catheter; a therapy delivery element operably associated with the elongate catheter; a stimulus delivery element operably associated with the elongate catheter; a stimulation source in communication with the stimulus delivery element, wherein application of a stimulus via the stimulus delivery element causes target tissue to contract on the elongate catheter to center the elongate catheter in the target tissue; and a therapy source in communication with the therapy delivery element, wherein application of the therapy denervates nerve tissue of the target tissue.

Example 13. The system for denervation according to Example 12, further comprising two stimulus delivery elements formed on the elongate catheter.

Example 14. The system for denervation according to Example 12, further comprising a second stimulus delivery element formed on a guide catheter.

Example 15. The system for denervation according to Example 12, wherein the stimulus delivery element is formed on a guide wire.

Example 16. The system for denervation according to Example 12, wherein the therapy source and the stimulation source are a single electrical generator.

Example 17. The system for denervation according to Example 12, wherein a therapy source modality is selected from the group consisting of monopolar or bipolar radio frequency (RF), microwave, ultrasound, chemical, cryogenic, and combination thereof.

Example 18. The system for denervation according to Example 12, wherein a stimulation source modality is selected from the group consisting of bipolar or monopolar alternating current (AC), a direct current (DC), pulsed AC or DC pulses, biphasic pulses, multiphasic pulses, chemical, and combinations thereof.

Example 19. The system for denervation according to Example 12, further comprising a shape memory portion configured to alter a shape of the elongate catheter to a helical configuration such that the therapy delivery element is in contact with the target tissue.

Example 20. The system for denervation according to Example 19, wherein the therapy element is composed of one or more monopolar RF electrodes formed on the elongate catheter such that alteration of the shape contacts the electrodes on the target tissue in spaced relation to each other.

Example 21. The system for denervation according to Example 12, further comprising a balloon, wherein inflation of the balloon secures the elongate catheter relative to the target tissue.

Example 22. The system for denervation according to Example 21, wherein the therapy delivery element is a plurality of RF electrodes formed on the balloon.

Example 23. The system for denervation according to Example 21, wherein the therapy delivery element is a microwave antenna within the balloon.

Example 24. The system for denervation according to Example 21, wherein the therapy delivery element is a plurality of ultrasound transducers formed on the balloon.

Example 25. The system for denervation according to Example 21, wherein the therapy delivery element is one or more needles for injection of a chemical or cryoablation medium to the target tissue.

Example 26. The system for denervation according to Example 21, further comprising a computer including a memory storing thereon an application that when executed by a processor: causes the stimulation element to apply a stimulus proximate the target tissue to contract muscle fibers of the target tissue; causes the therapy element to apply therapy to the target tissue; and ceases application of the therapy to cease upon determination that the target tissue has been successfully denervated.

Further disclosed herein is the subject-matter of the following clauses:
1. A system for denervation of target tissue comprising:
   an elongate catheter;
   a therapy delivery element operably associated with the elongate catheter;
   a stimulus delivery element operably associated with the elongate catheter;
   a stimulation source in communication with the stimulus delivery element, wherein application of a stimulus via the stimulus delivery element causes target tissue to contract on the elongate catheter to center the elongate catheter in the target tissue; and
   a therapy source in communication with the therapy delivery element, wherein application of the therapy denervates nerve tissue of the target tissue.
2. The system for denervation according to clause 1, further comprising two stimulus delivery elements formed on the elongate catheter.
3. The system for denervation according to clause 1 or 2, further comprising a second stimulus delivery element formed on a guide catheter.
4. The system for denervation according to any one of clauses 1 to 3, wherein the stimulus delivery element is formed on a guide wire.
5. The system for denervation according to any one of clauses 1 to 4, wherein the therapy source and the stimulation source are a single electrical generator.
6. The system for denervation according to any one of clauses 1 to 5, wherein a therapy source modality is selected from the group consisting of monopolar or bipolar radio frequency (RF), microwave, ultrasound, chemical, cryogenic, and combination thereof.
7. The system for denervation according to any one of clauses 1 to 6, wherein a stimulation source modality is selected from the group consisting of bipolar or monopolar alternating current (AC), a direct current (DC), pulsed AC or DC pulses, biphasic pulses, multiphasic pulses, chemical, and combinations thereof.
8. The system for denervation according to any one of clauses 1 to 7, further comprising a shape memory portion configured to alter a shape of the elongate catheter to a helical configuration such that the therapy delivery element is in contact with the target tissue.
9. The system for denervation according to clause 8, wherein the therapy element is composed of one or more monopolar RF electrodes formed on the elongate catheter such that alteration of the shape contacts the electrodes on the target tissue in spaced relation to each other.
10. The system for denervation according to any one of clauses 1 to 9, further comprising a balloon, wherein inflation of the balloon secures the elongate catheter relative to the target tissue.
11. The system for denervation according to clause 10, wherein the therapy delivery element is a plurality of RF electrodes formed on the balloon.
12. The system for denervation according to clause 10, wherein the therapy delivery element is a microwave antenna within the balloon.
13. The system for denervation according to clause 10 or 12, wherein the therapy delivery element is a plurality of ultrasound transducers formed on the balloon.
14. The system for denervation according to clause 10 or 12, wherein the therapy delivery element is one or more needles for injection of a chemical or cryoablation medium to the target tissue.
15. The system for denervation according to any one of clauses 10 to 14, further comprising a computer including a memory storing thereon an application that when executed by a processor:
   causes the stimulation element to apply a stimulus proximate the target tissue to contract muscle fibers of the target tissue;
   causes the therapy element to apply therapy to the target tissue; and
   ceases application of the therapy to cease upon determination that the target tissue has been successfully denervated.
16. A method of performing a therapeutic procedure, comprising:
   navigating a therapeutic device to target tissue;
   applying a first stimulus to the target tissue via a stimulus delivery element to contract the target tissue about the therapeutic device; and
   applying a first therapy to the target tissue contracted about the therapeutic device to denervate the target tissue.
17. The method according to clause 16, further comprising applying a second stimulus to the target tissue via the stimulus delivery element following the application of therapy to the target tissue.
18. The method according to clause 17, further comprising observing the application of the first and second stimulus to assess the contraction of the target tissue.
19. The method according to clause 18, wherein the therapy is applied following confirmation of contraction of the target tissue following application of the first stimulus.
20. The method according to clause 18, further comprising determining a successful denervation when no contraction or contraction below a threshold of the target tissue is observed from the second stimulus.
21. The method according to clause 18, further comprising applying a second therapy when a contraction above a threshold is observed of the target tissue from the second stimulus.
22. The method according to clause 16, wherein the first stimulus is applied during the application of the first therapy.
23. The method according to clause 22, further comprising observing the application of the first stimulus and the first therapy, to assess the contraction of the target tissue.

24. The method according to clause 23, further comprising stopping application of the first therapy upon observing a relaxation of the target tissue contracted by the application of the first stimulus, wherein relaxation of the target tissue indicates a successful denervation of the target tissue.

25. The method according to clause 16, wherein a first stimulation modality is selected from the group consisting of bipolar or monopolar alternating current (AC), a direct current (DC), pulsed AC or DC pulses, biphasic pulses, multiphasic pulses, chemical, and combinations thereof.

26 The method according to clause 16, wherein a first therapy modality is selected from the group consisting of monopolar or bipolar radio frequency (RF), microwave, ultrasound, chemical, cryogenic, and combination thereof.

1. A system for denervation of target tissue comprising:
an elongate catheter;
a therapy delivery element operably associated with the elongate catheter;
a stimulus delivery element operably associated with the elongate catheter;
a stimulation source in communication with the stimulus delivery element, wherein application of a stimulus via the stimulus delivery element causes target tissue to contract on the elongate catheter to center the elongate catheter in the target tissue; and
a therapy source in communication with the therapy delivery element, wherein application of the therapy denervates nerve tissue of the target tissue.

2. The system for denervation according to claim 1, further comprising two stimulus delivery elements formed on the elongate catheter.

3. The system for denervation according to claim 1 or 2, further comprising a second stimulus delivery element formed on a guide catheter.

4. The system for denervation according to any one of claims 1 to 3, wherein the stimulus delivery element is formed on a guide wire.

5. The system for denervation according to any one of claims 1 to 4, wherein the therapy source and the stimulation source are a single electrical generator.

6. The system for denervation according to any one of claims 1 to 5, wherein a therapy source modality is selected from the group consisting of monopolar or bipolar radio frequency (RF), microwave, ultrasound, chemical, cryogenic, and combination thereof.

7. The system for denervation according to any one of claims 1 to 6, wherein a stimulation source modality is selected from the group consisting of bipolar or monopolar alternating current (AC), a direct current (DC), pulsed AC or DC pulses, biphasic pulses, multiphasic pulses, chemical, and combinations thereof.

8. The system for denervation according to any one of claims 1 to 7, further comprising a shape memory portion configured to alter a shape of the elongate catheter to a helical configuration such that the therapy delivery element is in contact with the target tissue.

9. The system for denervation according to claim 8, wherein the therapy element is composed of one or more monopolar RF electrodes formed on the elongate catheter such that alteration of the shape contacts the electrodes on the target tissue in spaced relation to each other.

10. The system for denervation according to any one of claims 1 to 9, further comprising a balloon, wherein inflation of the balloon secures the elongate catheter relative to the target tissue.

11. The system for denervation according to claim 10, wherein the therapy delivery element is a plurality of RF electrodes formed on the balloon.

12. The system for denervation according to claim 10, wherein the therapy delivery element is a microwave antenna within the balloon.

13. The system for denervation according to claim 10 or 12, wherein the therapy delivery element is a plurality of ultrasound transducers formed on the balloon.

14. The system for denervation according to claim 10 or 12, wherein the therapy delivery element is one or more needles for injection of a chemical or cryoablation medium to the target tissue.

15. The system for denervation according to any one of claims 10 to 14, further comprising a computer including a memory storing thereon an application that when executed by a processor:
causes the stimulation element to apply a stimulus proximate the target tissue to contract muscle fibers of the target tissue;
causes the therapy element to apply therapy to the target tissue; and
ceases application of the therapy to cease upon determination that the target tissue has been successfully denervated.
